(19) United States
(12) Patent Application Publication
(10) Pub. No.: US 2017/0165043 A1
(43) Pub. Date: Jun. 15, 2017

(54) PARABOLIC EYEGLASSES PESSARY

(71) Applicant: EURL CORNIER, Paris (FR)

(72) Inventor: Edgard CORNIER, Paris (FR)

(21) Appl. No.: 15/373,970

(22) Filed: Dec. 9, 2016

(30) Foreign Application Priority Data
Dec. 11, 2015 (FR) 1562175

(51) Int. Cl. A61F 2/00; A61F 6/08

(52) U.S. Cl. CPC A61F 2/005; A61F 6/08; A61F 2/0009; A61F 2230/0006

(57) ABSTRACT

Parabolic pessary characterized in that it is composed of a ring-shaped component (1) that is open in a lunette shape or closed in a ring shape, with a raised distal posterior part (4) of cube shape or flat shape, having the role of supporting the uterus and two lateral branches (5, 5′) that are thinner than the distal part (4), and a proximal anterior part (6) that is positioned at the median part of the feminine urethra, this ring-shaped component (1) having an upper face (2) with a paraboloid shape that adapts to the pelvic paraboloid, and a posterior face (3) having the shape of a more-open paraboloid having, at its median part, a thicker part (8) allowing it to bear on the pelvic floor.

The invention herein concerns a parabolic eyeglasses pessary intended to be positioned in the vagina of a patient, to maintain the organs of the pelvic cavity, particularly the uterus and the vagina in position above the pelvic floor, and to correct urinary incontinence from effort.

Pessaries are medical devices used to treat prolapses and/or urinary incontinence in women, that have been known for many years, and have long been considered to be palliative therapeutic means in cases of contra-indications or refusal of surgery, principally in the case of women of very advanced age.

However, pessaries are now drawing interest again because of the failures and complications of surgical procedures using non-resorptive synthetic wicks to treat impairments of the vaginal wall.

The Food and Drug Administration (2012) has even published advisories of contra-indications for the surgical implantation of this type of prosthesis in the wall of the vagina.

It should also be noted that, according to the recommendations of Canadian specialists in 2013 (Obstet Gynaecol Can 2013; 35(7): 675-676), a pessary “should be considered for all women experiencing a symptomatic prolapse and/or urinary incontinence from effort”.

Despite their recommendations, these specialists were not able to define the characteristics of an “ideal” pessary, and ten different models of pessary illustrate the above-mentioned article, with no specific recommendation with respect to cases encountered.

They also observed that, despite this diversity, the global rates of usage of pessaries falls to less the 50%—and, even, less than 20%—in cases of urinary incontinence.

There are multiple reasons for this discontinuation of use, and one can cite—as an example—poor hygiene due to too many re-uses, due to the cost of pessaries or to descent of the pessary, which is often a poor fit for the anatomy of each patient.

In addition, to prevent the risk of descent, the model chosen is often too big, which causes pains, bleedings through erosion and even superinfections when the pessary is left fitted for too long a time.

With regard to this, it should be noted that a pessary is frequently only replaced by medical personnel.

Yet the Canadian writers emphasize that, when the pessary is properly adapted to the patient's anatomy, it can be used as long as necessary without repercussions, and it does not constitute an impediment to sex life, which it often aids.

In 2007, the French National College of Gynecologists published observations in which the fall in use of the pessary was acknowledged, but it concluded that the use of this medical appliance should be reconsidered in a positive light, because of the disadvantages of the surgery that is offered as first-ranking proposal for treating problems of prolapse of the pelvic organs and/or urinary incontinence: this surgery does indeed pose problems in connection with non-negligible side effects, risks of failures, and special complications (erosion, permanent pains, and sexual problems because of complications from weaved prostheses surgically placed in the vaginal walls).

There are currently around one hundred different models of pessary, of which the most-widely used are the ring pessary, the donut pessary, the DISH pessary and cubic pessaries that have become available more recently, among which one can mention the GYN&Cube pessary of pyramidal shape, and the cubic pessary that is of cubic shape with concave faces.

The ring pessary consists of a rubber or silicon ring that is positioned by folding it towards the base of the vagina and the passing it behind the pubic symphysis; this pessary has proved to be effective in the case of small prolapses without urinary incontinence.

The donut pessary consists of a thicker ring, and is designed for the treatment of bigger uterine prolapses; such a ring is difficult to insert into the vagina, because it does not bend, and so is difficult to bear for narrow vaginae; plus, it does not treat urinary continence from effort.

The DISH pessary is a silicon device equipped with a tongue that is intended to position the neck of the urinary bladder and the sub-vesical urethra in abdominal situation, in order to allow the sphincter of the urinary bladder the ability to prevent leaks under effort.

Cubic pessaries are intended to raise the uterus by pressing via suction effect on the walls of the vagina; once positioned in the base of the vagina, these pessaries have good effectiveness and are well tolerated, but are difficult to remove because of their suction effect on the vaginal walls; furthermore, they have no effect on urinary incontinence.

These pessaries have the disadvantage of only having a partial effectiveness on each of the different compartments of the genito-urinary sphere affected by specifically-feminine problems such as urine loss in association with a vaginal or uterine prolapsed.

Therefore, it would be desirable to design and provide women affected by these problems with a pessary that is better adapted to the physiology of the pelvic equilibriums.

The invention herein is intended to propose such a pessary.

The current theory of pelvic equilibrium is based on the concept of “pelvic floor”; the organs of the pelvic cavity, such as the neck of the urinary bladder, the walls of the vagina and the rectum, are supposed to rest on this pelvic floor (Kamina Anatomie Humaine—Maloine Ed—2012).

According to this theory, the uterus is maintained above the pelvic visco-elastic space by anatomic structures called ligaments: round ligaments, parameters and the uterosacral ligaments.

The mechanism of the effectiveness or inadequacies of the various pessaries currently commercially available is not clearly explained by this physiology: according to the French National College of Gynecologists and Obstetricians [Collège National des Gynécologues et Obstétriciens Français], pessaries use the pubo-rectal clusters of levitators as a bearing point; in the case of cubic pessaries, which are located and maintained at a distance from the pubo-rectal clusters of levitators, it is supposed to act through the friction on the posterior fornix (that and the pessary is) to maintain(ed) his position and lift the uterus.

The College claims that discontinuing the use of the pessary is not justified: It is prescribable for 60% of patient cases, but only 25% will use it on an extended basis, because of the above-mentioned disadvantages.

Recent studies resulted in a new approach to the equilibrium of the pelvic visco-elastic medium.

These studies apply directly to the designing of a more physiology-friendly pessary that would be better accepted by women.

Unpublished document US 14 924 200 notably describes a process for modeling the direction of the incident abdominal pressures towards the feminine pelvic cavity, and the direction of the pressures reflected in the pelvic space.

To enable such a modeling, this document studies the vectors of the pressures that propagate through the pelvic visco-elastic tissue.

Analysis of IRM 3D cube images shows that the pelvis can be assimilated to a paraboloid—defined by the sacrococcygeal concavity extending longitudinally through the pelvic floor—that reflects the vectors resulting from abdominal pressure.

But the geometry of this paraboloid is influenced by that of the pelvic floor, which can vary in length or be short at the urogenital cleft.

The paraboloid thus described returns the incident vectors resulting from abdominal pressure at varying height (above or below the pubis).

One can thus establish that everything is proceeding satisfactorily if the paraboloid is sufficiently long to return the incident vectors upwards; contrariwise, if it the paraboloid is too short, the pressure will direct the organs downwards (prolapse).

This analysis of the pressures that are reflected by the pelvic paraboloid in normal state also applies to a pessary in position when the pelvic floor is deficient, too short or too open: the pessary then corrects and completes the form of the paraboloid, to reflect the pressures in the right direction.

This approach has thus led to the designing—in accordance with the invention—a pessary having a parabolic shape that adapts to the pelvic paraboloid so as to extend it and direct the vector resulting from the reflected abdominal pressure, upwards and towards the front of the abdomen, namely the anterior abdominal wall and the osseous pubis.

Other recent studies described in the unpublished document EP 15 175 460.3 have enabled one to observe, in IRM images, that in continent women, the sub-vesical urethra has a zig-zag form that is not present in the urethras of incontinent women, and have thus suggested that the angle of the plication of the sub-vesical urethra has a crucial role in enabling urinary continence: the urethra folds onto itself when it is folded under effort, which prevents leaks and produces greater rectilinearity when the urinary bladder is full.

We thus had the idea—in accordance with the invention—of proposing a parabolic pessary that can bear on the area of plication of the urethra, and thus rise to approximately 25 mm above the feminine urinary meatus within the vagina.

In this context, the invention herein addresses a pessary of the above-mentioned type constituted by a ring-shaped component open like an inverted monocular eyeglasses or closed in a ring, made of a supple anallergic bio-compatible material, having a raised distal posterior part of cube shape, having the function of supporting the uterus, with two lateral branches that are thinner than the distal part, and one proximal anterior part.

These finer lateral branches enable the pessary to be gripped so as to facilitate its positioning.

According to the invention, the pessary has an upper face at which the raised distal posterior part of cube shape, and a posterior face.

The upper face has a parabolic shape that adapts to the pelvic paraboloid so as to extend it and direct the reflected abdominal pressure upwards and towards the front of the abdomen, namely the anterior abdominal wall and the osseous pubis.

The posterior face is of more-open paraboloid shape, and its posterior part has a thicker part, allowing it to bear on the pelvic floor.

The parabolic pessary according to the invention can thus integrate into the pelvic visco-elastic space, to redirect the reflected abdominal pressure upwards and towards the front, and not downwards.

According to another characteristic of the invention, the distal posterior part of the ring-shaped component is raised by approximately 20 to 30 mm in relation to the proximal anterior part.

This characteristic enables the uterus to be helped to no longer descend under the stress of abdominal pressure; it thus remains above the pelvic visco-elastic space, so as to be projected upwards by the force due to its domed shape.

According to one particularly-advantageous characteristic of the invention, the upper face of the ring-shaped component has, at its anterior proximal extremity, a thicker part forming a button having the function of pressing on either side of the urethra, at its plication, to enable the maintenance of the urethral zig-zag and the medio-urethral angle of closure.

Because of this configuration, the bearing pressure created by the abdominal pressure is transmitted to this button on the parabolic pessary, to ensure the maintenance of the urethral zig-zag and prevent the opening of the medio-urethral angle of closure.

According to another characteristic of the invention, the ring-shaped component is entirely or partially hollow, so as to instill the pessary with an elasticity, so as to enable its positioning while retaining sufficient strength.

According to another advantageous characteristic of the invention, the parabolic pessary is closed by a membrane having the function of forming an anti-conceptional barrier.

The parabolic pessary according to the invention can, of course, be produced in a series of different dimensions, and made of different bio-compatible and anallergic materials being sufficiently supple and sterilizable.

However, the pessary will preferably be made of a sterilizable silicon.

The characteristics of the parabolic pessary that is the subject of the invention will be described in greater detail, with reference to the appended non-limitative drawings, in which:

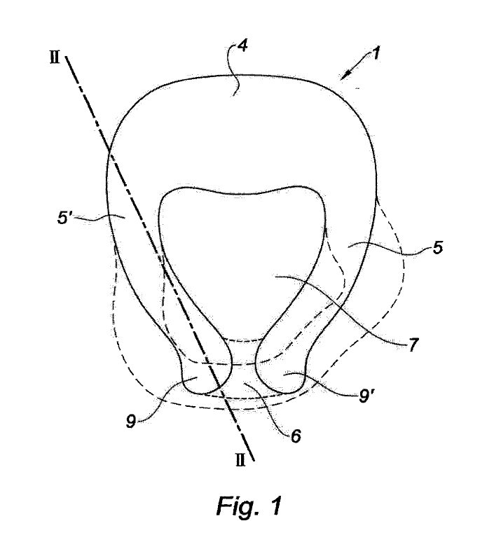

FIG. 1 is a top view of the upper face of the pessary; the dotted lines represent a configuration variant;

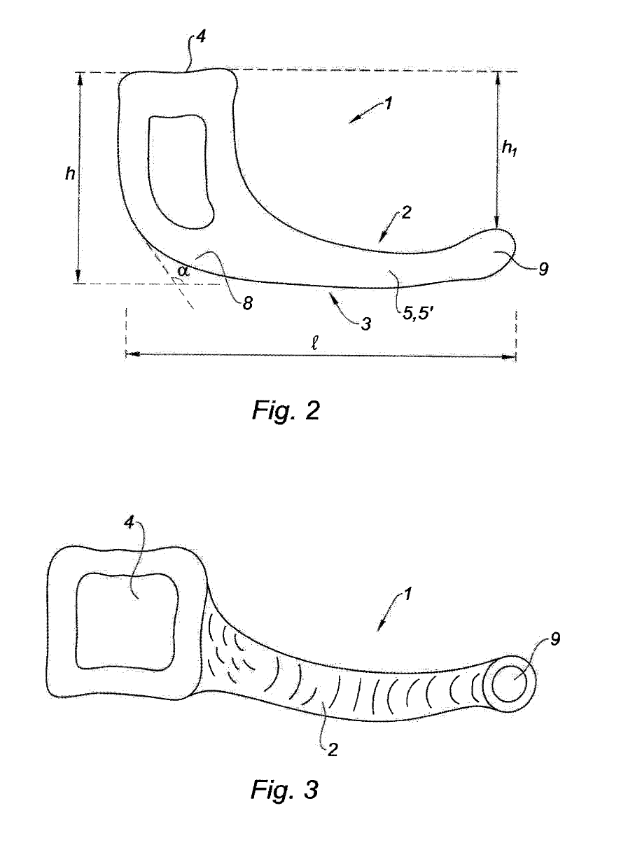

FIG. 2 is a profile cross-section of the pessary, according to axis II-II of FIG. 1;

FIG. 3 is a parasagital profile cross-section of the pessary illustrated in FIG. 1;

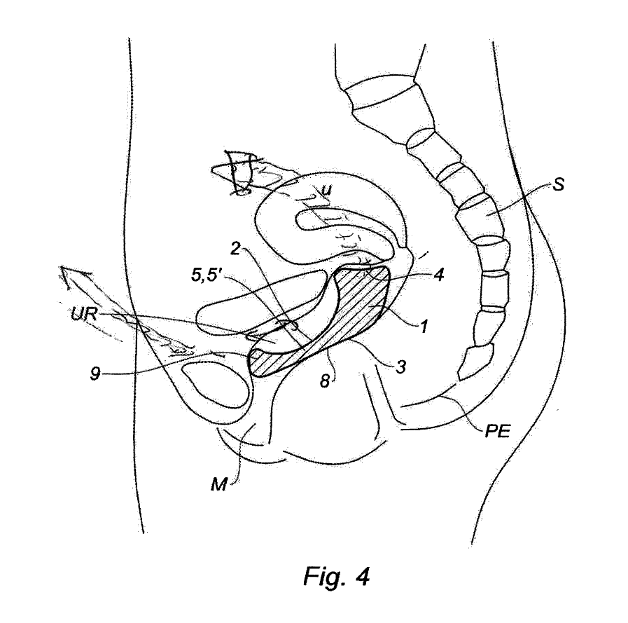

FIG. 4 is a diagram illustrating the parabolic pessary positioned in the pelvic cavity.

In the illustrations, the parabolic pessary is a ring-shaped component that is open or closed at the front, as a monoculmar eyeglasses having an upper face 2 and a posterior face 3.

According to FIG. 1, the ring-shaped component that is open or closed at the front, 1, has a raised distal posterior part, 4, of cube shape or flat, such as illustrated in FIG. 2, together with two lateral branches, 5, 5′, that are thinner than the distal part, 4, and are—possibly—closed by a proximal anterior part, 6.

It should be noted that the central aperture, 7, of the ring-shaped component, 1, can be closed by a membrane not having been illustrated, that has the role of forming an anti-conceptual barrier.

According to FIG. 4, the raised distal posterior part, 4, of cube shape, is positioned under the uterus, U, which it has the role of supporting.

According to FIGS. 2 and 4, the upper face, 2, of the ring-shaped component, 1, has a paraboloidal shape that adapts to the pelvic paraboloid defined by the sacrum, S, and the pelvic floor, PE, so as to extend it and direct the reflected abdominal pressure upwards and towards the front of the abdomen, namely the anterior abdominal wall and the osseous pubis.

According to FIG. 2, the posterior face, 3, of the ring-shaped component, 1, is of more-open paraboloid shape, and its median part has a greater thickness, 8, allowing it to bear on the pelvic floor, PE, such as illustrated in FIG. 4.

In a manner not illustrated in the Figures, a vibrating motor can be inserted into the pessary at this thicker part.

According to FIGS. 1 to 4, the upper face, 2, of the ring-shaped component, 1, has—at its proximal extremity—a thicker part forming a button, 9, that has the function of bearing on either side of the urethra, UR.

According to the example of implementation illustrated in FIG. 1, this button has two parts, 9, 9′, having the role of bearing either side of the urethra, to enable maintenance of this angle and the urethral zig-zag.

According to FIG. 2, the ring-shaped component, 1, has—on average—a height (h) of approximately 20 to 40 mm and a width (1) of around 25 to 50 mm. In addition, the distal posterior part, 4, is raised in relation to the thicker part forming button, 9, by a height (h1) of approximately 20 to 30 mm.

It should be noted that the essential characteristics of the parabolic pessary addressed by the invention are in connection with the heightening in cube shape or flat shape of the distal posterior part, 4, with the paraboloidal shape of the upper face, 2, and the posterior face, 3, and the presence of the sub- or para-urethral thicker part forming a button, 9, 9′, closed ring or open, eyeglasses like.

However, the proximal anterior part, 6, can be relatively narrow, such as illustrated by the solid lines in FIG. 1, or wider, such as illustrated by dotted lines in the said FIG. 1, without this falling outside the scope of the invention.

### NOMENCLATURE

1 Ring-shaped component
2 Upper face
3 Posterior face
4 Distal posterior face
5, 5′ Lateral branches
6 Proximal anterior part
7 Central oval aperture
8 Thicker part forming a heel
9, 9′ Thicker part forming a button

What is claimed is:

1. Parabolic pessary intended to be positioned in the vagina of a patient, to maintain the organs of the pelvic cavity, particularly the uterus and the vagina in position above the pelvic floor, and correct urinary incontinence from effort.
characterized in that
it is composed of a ring-shaped component that is open in a lunette eyeglasses shape or closed in a ring shape, made of a supple anallergic biocompatible material (1) with a raised distal posterior part (4) of cube shape (or flat shape), having the role of supporting the uterus and two lateral branches (5, 5′) that are thinner than the distal part (4), and a proximal anterior part (6), with this ring-shaped or opened eyeglasses shape component (1) having an upper face (2) with a paraboloid shape that adapts to the pelvic paraboloid, and a posterior face (3) having the shape of a more-open paraboloid having, at its (median) posterior part, a thicker part (8) allowing it to bear on the pelvic floor.

2. Parabolic pessary in accordance with claim 1,
characterized in that
the upper face (2) of the ring-shaped or opened eyeglasses shape component (1) has, at its anterior proximal extremity, a thicker part forming a button (9, 9′) having the function of pressing (on the urethra, or) on either side of the urethra, to enable the maintenance of the urethral zig-zag and the medio-urethral angle of closure.

3. Parabolic pessary according to claim 1,
characterized in that
the distal posterior part (4) of the ring-shaped component (1) is raised by approximately 20 to 30 mm in relation to the proximal anterior part (6).

4. Parabolic pessary according to claim 1,
characterized in that
the ring-shaped component (1) is totally or partially hollow.

5. Parabolic pessary according to claim 1,
characterized in that
it is closed by a membrane having the function of forming an anti-conceptual barrier.

6. Parabolic pessary according to claim 1,
characterized in that
it is made of a sterilizable silicon.
